(19) United States
(12) Patent Application Publication
(10) Pub. No.: US 2009/0222018 A1
(43) Pub. Date: Sep. 3, 2009

(54) Instrument for Introducing Solid Fillers into the Interior of a Bone

(75) Inventor: Bruno Sidler, Menziken (CH)

Correspondence Address: FISH & ASSOCIATES, PC;ROBERT D. FISH, 2603 Main Street, Suite 1000, Irvine, CA, 92614-6232, US

(73) Assignee: AKROSS AG, Schaffhausen (CH)

(21) Appl. No.: 12/282,315

(22) PCT Filed: Mar. 19, 2007
(86) PCT No.: PCT/EP2007/052562

§ 371 Date: May 11, 2009

(30) Foreign Application Priority Data
Mar. 20, 2006 (EP) 06111415.3

(51) Int. Cl. A61B 17/58

(52) U.S. Cl. 606/93

(57) ABSTRACT

The instrument (1) for introducing solid fillers (2) into the interior of a bone comprises a cannula (3) and a handle (4) connected to the latter, wherein a reservoir (5) is mounted displaceably in the handle (4), and wherein the reservoir (5) has a plurality of chambers (5a) for receiving solid fillers (2), the chambers (5a) being arranged in the chamber (5) in such a way that, by displacement of the reservoir (5), the chambers (5a) can be positioned one after another in a dispensing position (A) relative to the cannula (3), such that the fillers (2) of the chamber (5b) located in the dispensing position (A) can be delivered to the cannula (3).

The present invention relates to an instrument for the delivery of solid fillers into a inner space of a bone in accordance with the preamble of claim 1.

Document WO2005/094735, whose disclosure is herewith included herein by reference, describes a filler, a delivery device as well as a method for the formation of a support structure in an inner space of a bone. In this respect, a plurality of support bodies are introduced into an inner space of a bone, in particular into a vertebral body.

Reference US 2005/0228397 discloses an instrument for the delivery of bone cement, in particular liquid bone cement, into the inner space of a vertebra. This instrument has the disadvantage that it is not suitable for the delivery of solid fillers. The instrument is moreover difficult to operate so that it is difficult for a surgeon to fill an inner space of a bone reliably and reproducibly.

It is the object of the present invention to propose an instrument for the delivery of solid fillers into an inner space of a bone, said instrument permitting a particularly simple, secure and reliable delivery.

This object is satisfied by an instrument having the features of claim 1. Dependent claims 2 to 15 relate to further, advantageously designed instruments.

The object is in particular satisfied using an instrument including a cannula as well as a handle connected thereto, with a reservoir being mounted displaceably in the handle and with the reservoir having a plurality of chambers for the reception of solid fillers, with the chambers being arranged in the reservoir such that the chambers can be sequentially positioned in a dispensing position with respect to the cannula by displacement of the reservoir so that the fillers can be delivered to the chamber of the cannula respectively located in the dispensing position.

The instrument in accordance with the invention has the advantages that it is very simple to operate, that the instrument can be connected very easily to the inner space of the bone and that the fillers can be delivered very simply and reliably to the inner space of the bone. In addition, a surgeon can himself determine in a simple manner how many fillers are delivered to the inner space of the bone since the instrument includes a reservoir loaded with fillers and the fillers are arranged in groups of, for example, 2, 3, 5 or 10 pieces in a chamber of the reservoir so that in each case such a group of fillers arranged sequentially can be supplied to the inner space of the bone. After the delivery of such a group of fillers, a decision can be made whether a further group should be delivered, with the procedure of the delivery of groups of fillers being able to be repeated until the vertebral body has been sufficiently filled with fillers.

The instrument in accordance with the invention in particular allows a particularly simple access to the spinal column in that a short, and above all slim and rigid cannula is used which is set at the vertebral body. The handle of the instrument is usually fixedly connected to the cannula so that the cannula can be aligned and placed at the vertebral body to be treated by a corresponding holding of the handle. This permits a simple and secure operation of the instrument. This cannula, which is usually provided with a trocar during the first introduction, has the great advantage that, on the introduction through the sensitive back musculature, it is precisely these muscles which are spared to a maximum. The patient thereby becomes mobile faster again after the operation. He is also spared pronounced scar formation in the region of the back muscles.

The instrument has a rigid cannula which is preferably fixedly connected to the handle. In a preferred embodiment, the handle is made such that it can be held by the left hand. In a preferred embodiment, the instrument has a push button with which the reservoir can be moved to position fillers in a dispensing position. In a preferred embodiment, this push button can be acquitted using the thumb of the left hand. A plunger is preferably held in the right hand. This plunger is introduced into the handle, with the fillers present in the dispensing position simultaneously being expelled from the reservoir or from the reservoir chamber by the plunger tip and the fillers thereafter being expelled into the inner space of the bone via the cannula. The manual operation of the plunger has the advantage that the expulsion force to be applied on the introduction of the fillers into the inner space of the bone is felt by surgeons so that, for example, too large an expulsion force which could damage the bone can be avoided. The manual operation of the plunger additionally has the advantage that the fillers present in the inner space of the bone can be positioned, for example when an image producing process, for example an X-ray machine, is used during the introduction of the fillers.

The reservoir can be made in very varied manners, for example also in the form of a bar-shaped reservoir extending in the longitudinal direction or of a circular reservoir made as a drum.

The instrument is preferably supplied together with a trocar which in particular serves for the opening of the inner space of the bone. The instrument is preferably supplied with a trocar extending through the cannula and fastened to the handle, with the reservoir additionally already being filled with fillers so that the essential components are supplied as a single unit.

The instrument in accordance with the invention makes it possible for a surgeon to carry out all the major process steps using two hands without any additional help so that the instrument can be operated very easily, securely and reliably.

The instrument in accordance with the invention is in particular suitable for the filling of vertebral bodies. The instrument in accordance with the invention, however, allows any inner spaces of bones to be provided with fillers, for example also bones damaged or weakened in the most varied manner, for example due to trauma, infection, wear, tumor growth or degenerative diseases such as osteoporosis. In older people, in particular osteoporosis, that is the degradation of the spongiosa, represents a problem because the load capability of the bone is thereby weakened, which has the result that bone fractures occur more frequently, above all at the spinal column, at the femoral neck and at the wrist. The instrument in accordance with the invention makes it possible to introduce fillers into such bones, with the introduction, if necessary, moreover enabling a disimpacting of a bone, for example of a collapsed vertebral body.

The invention will be described in detail in the following with reference to Figures, with the same reference numbers designating the same objects. There are shown:

FIG. 1 a perspective view of the instrument for the delivery of solid fillers into an inner space of a bone;

FIG. 2 a perspective view of the instrument from below;

FIG. 3 a side view of the instrument;

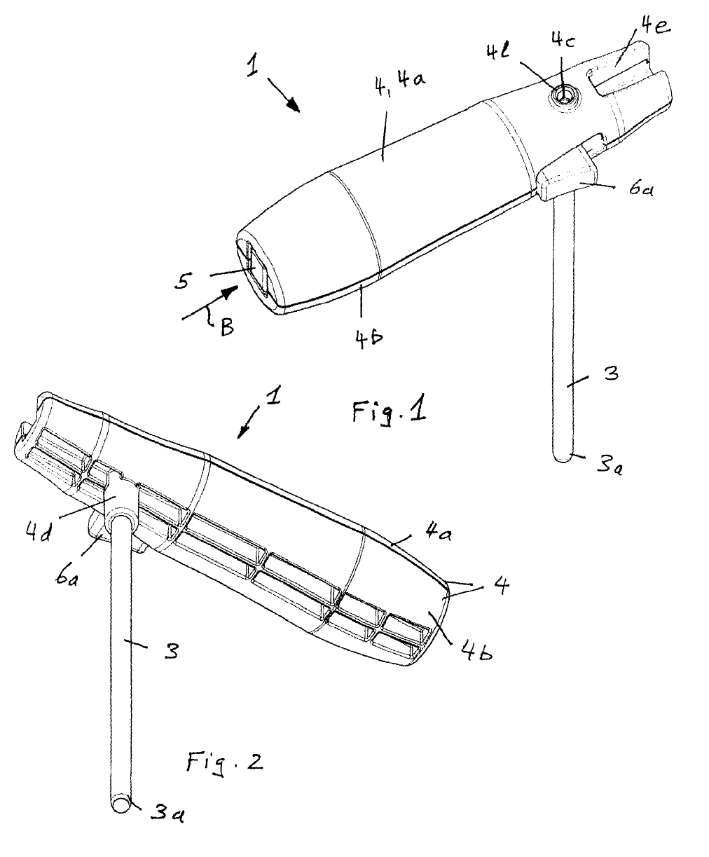

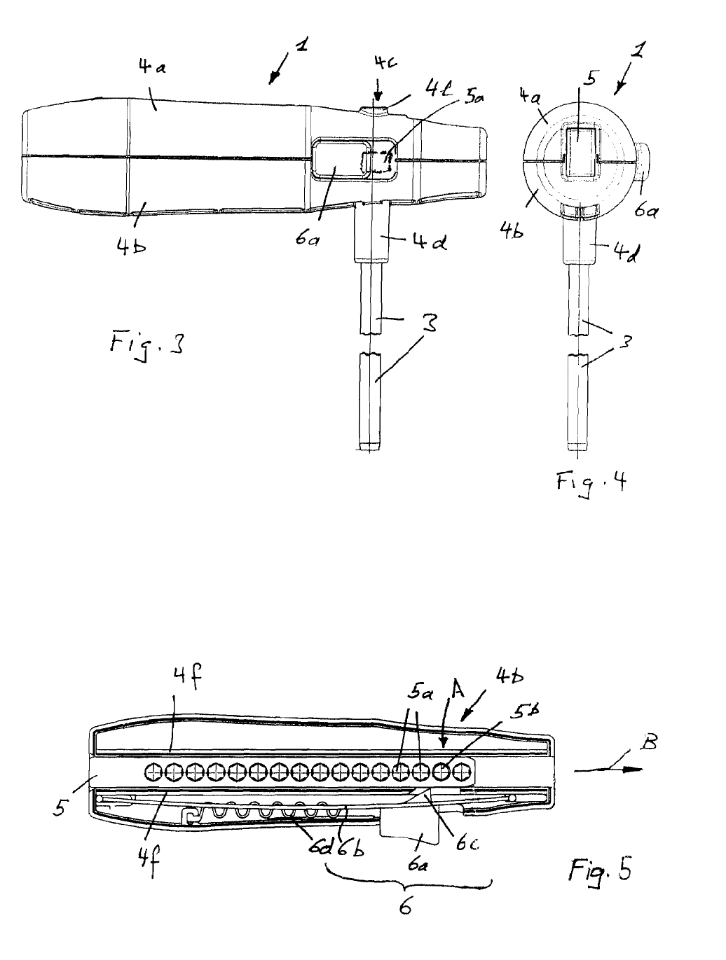

FIG. 4 a view of the end face of the instrument;

FIG. 5 an inner view of the lower half of the handle part of the instrument in accordance with FIG. 1;

FIG. 6 a detailed view of the left hand end section of the upper half of the handle part;

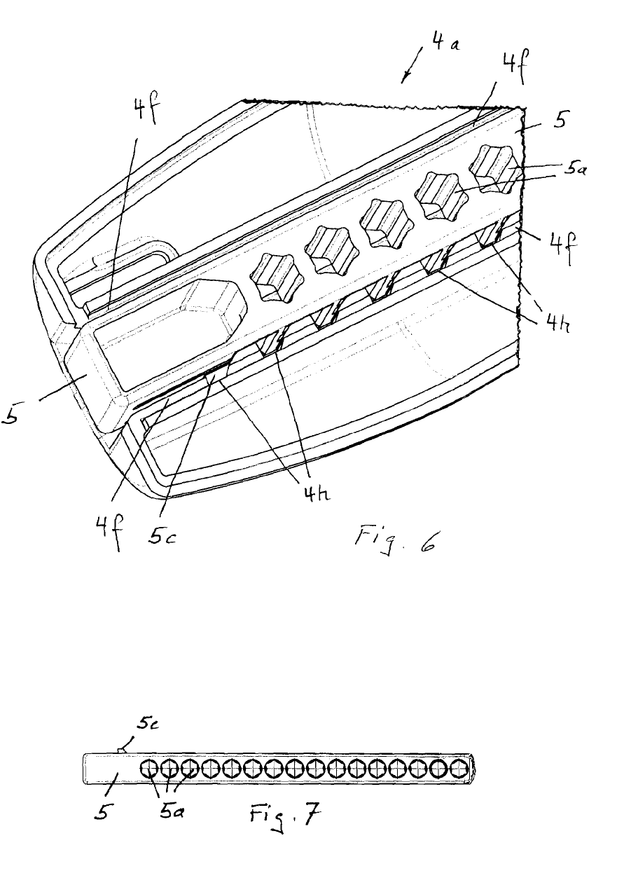

FIG. 7 a plan view of a reservoir;

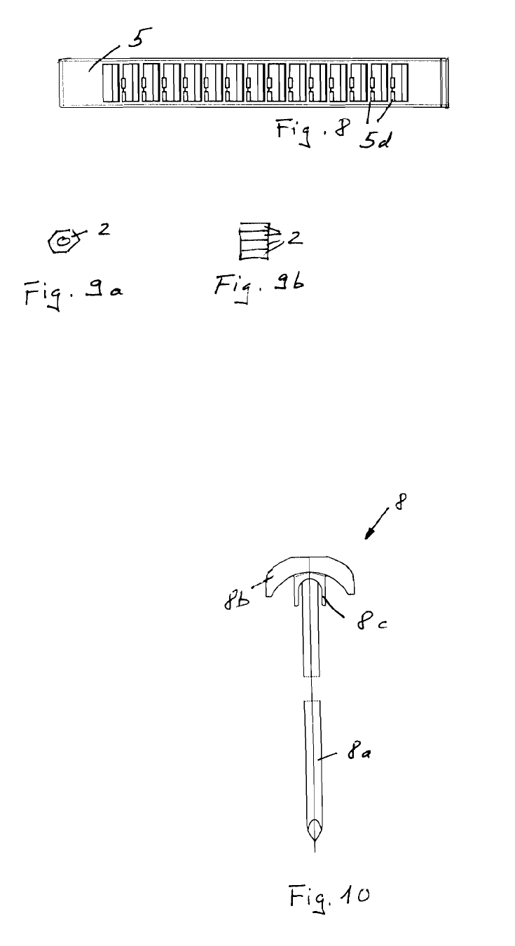

FIG. 8 a side view of the reservoir in accordance with FIG. 7;

FIG. 9a a plan view of a filler;

FIG. 9a a side view of a plurality of stacked fillers;

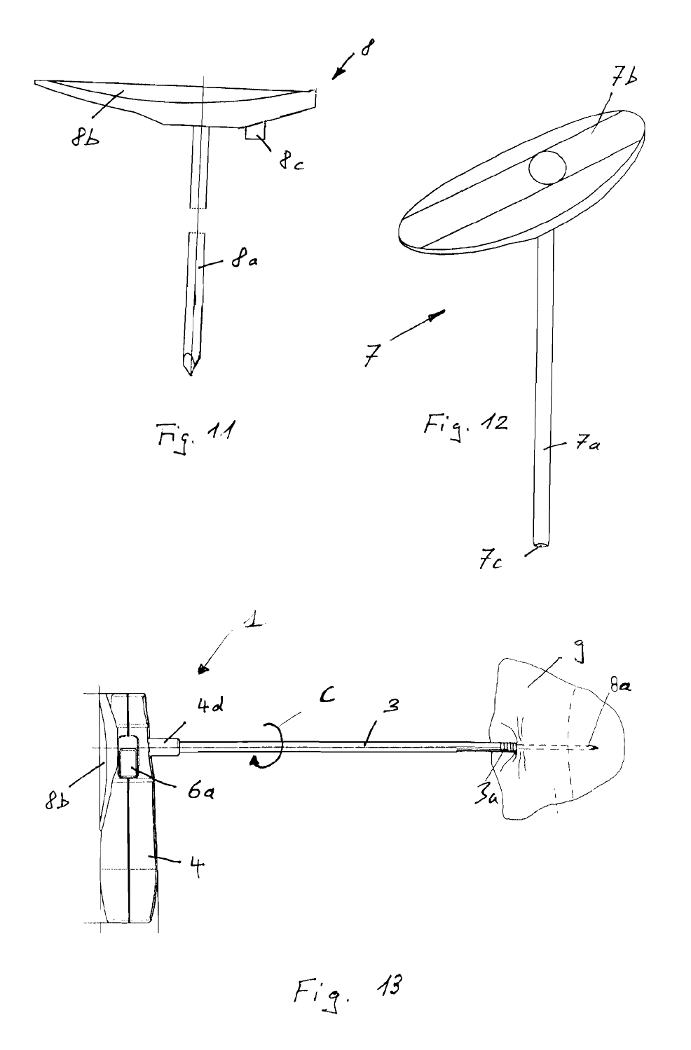

FIGS. 10, 11 views of a trocar;

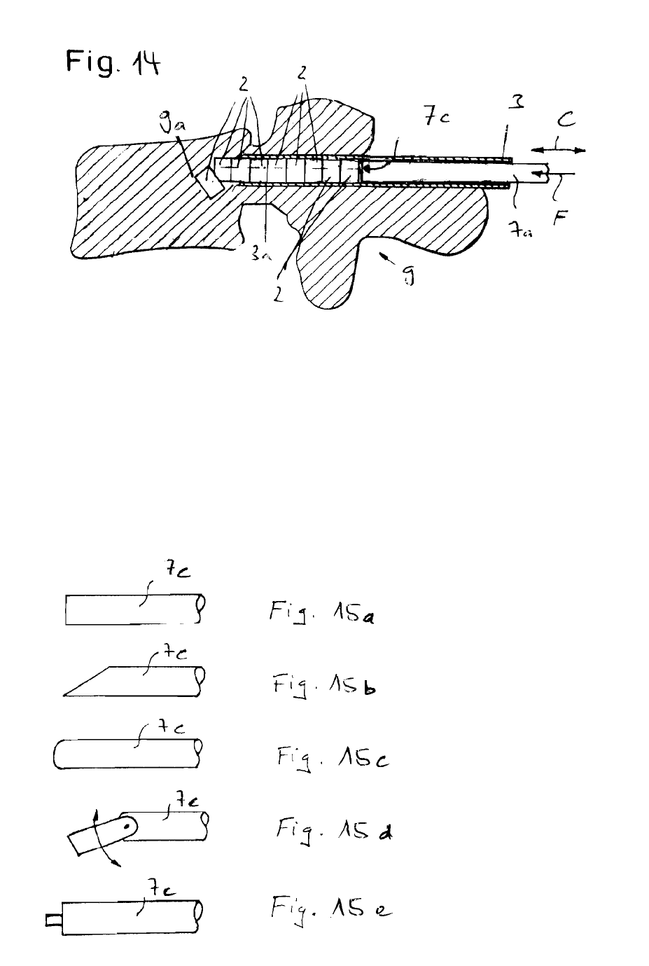

FIG. 12 a plunger;

FIG. 13 an instrument during the connection to a bone;

FIG. 14 a section through a vertebra during the introduction of the fillers;

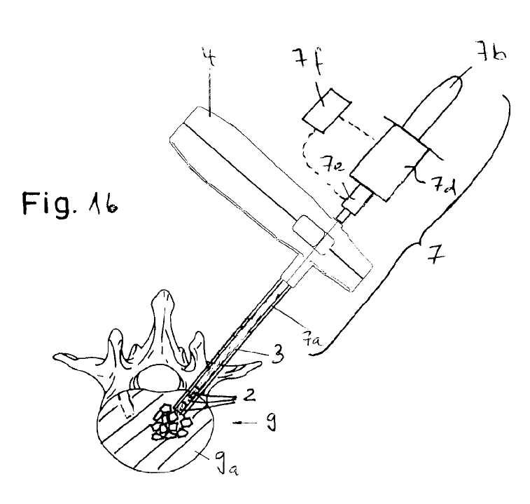

FIGS. 15a-15e a plurality of differently made plunger tips;

FIG. 16 the instrument in combination with an actively driven plunger;

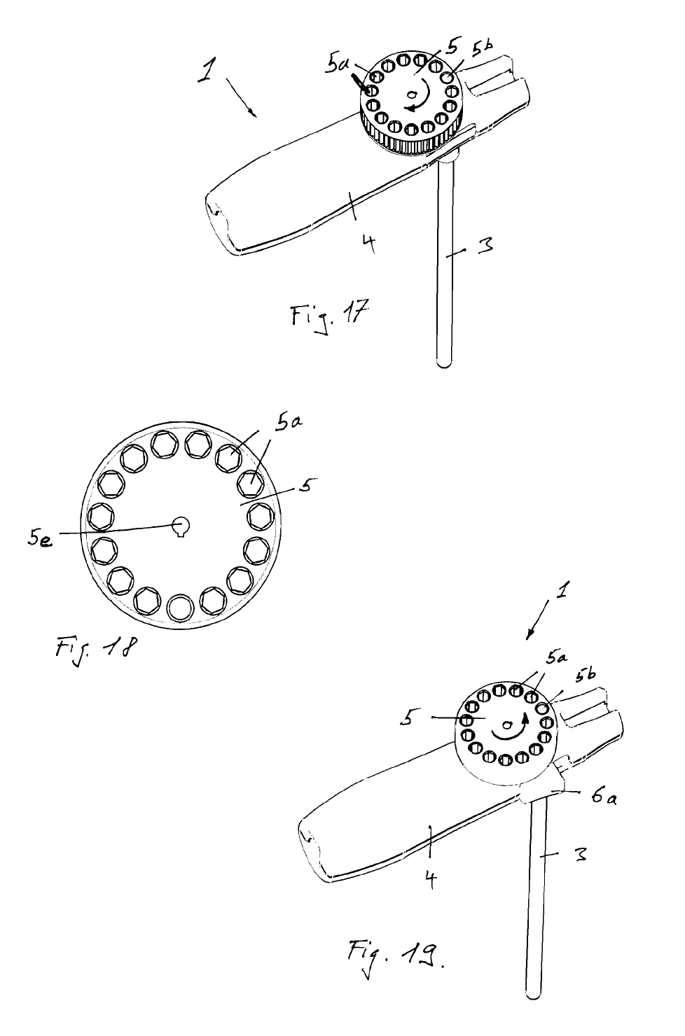

FIG. 17 a perspective view of a further embodiment of an instrument with a rotatable reservoir;

FIG. 18 a perspective view of a rotatable reservoir;

FIG. 19 a perspective view of a further embodiment of an instrument with a rotatable reservoir.

FIGS. 1 to 4 show the same instrument 1 for the delivery of solid fillers into an inner space of a bone from different directions of view. The instrument 1 includes a handle 4 as well as a rigid cannula 3 which are fixedly connected to one another. The handle 4 includes an upper housing half 4a as well as a lower housing half 4b which are likewise fixedly connected to one another. The handle 4 moreover includes a holder 4d for the cannula 3 as well as an access opening 4c which is surrounded by a projecting sleeve 41. As can be seen from the side view in accordance with FIG. 3, the access opening 4c is arranged in a straight-line extension with respect to the cannula 3 so that the shaft 8a, 7a of a trocar 8 or of a plunger 7 can be introduced into the access opening 4c and can be introduced via the cannula 3 up to the cannula tip 3a and, if necessary, also beyond it. The cannula 3 is made rigid or stiff and is preferably made of a metal or of a firm plastic.

In the embodiment shown, a reservoir 5 is mounted in the interior of the handle 4, displaceable in the direction of movement B and extending in its longitudinal direction. The handle 4 additionally includes a feed part 6 with a push button 6a by whose actuation the reservoir 5 is displaceable by a defined path in the direction of movement B.

The instrument 1 shown is optimized for the left hand in that the handle 4 can be surrounded by the left hand such that the push button 6a can be actuated in the direction of displacement B. Thus, both the handle 4 can be held and the position of the reservoir 5 can be changed. Since the cannula 3 is fixedly connected to the handle 4, the tip of the cannula 3 can contact the vertebral body by a corresponding holding of the handle 4.

FIG. 5 shows an inner view of the lower half 4b of the handle 4. The lower handle half 4b has lateral guides 4f which extend in the direction of extent B and between which a reservoir 5 is displaceably mounted in the direction B. The reservoir 5 includes a plurality of chambers 5a, 5b arranged spaced apart in the direction B. One of the chambers, designated by 5b, is located directly above the cannula 3. This position of a chamber is designated as the dispensing position A since the chamber 5b is located directly above the cannula 3. The instrument 1 moreover includes a feed part 6 which, in the embodiment shown, includes a push button 6a, a spring 6b and an engagement nose 6c as well as a restoration spring 6d.

FIG. 8 shows a side view of the reservoir 5 with a plurality of grooves 5d spaced apart in the longitudinal direction B. These grooves 5d are aligned toward the engagement nose 6c in the arrangement shown in FIG. 5 so that, when the push button 6a is actuated, its engagement nose 6c engages into the groove 5b, with the push button 6a simultaneously being moved in the direction B so that the reservoir 5 is displaced in the direction B by a path defined such that the chamber 5a most closely following the chamber 5b moves into the dispensing position A.

FIG. 7 shows a plan view of the reservoir 5 with a plurality of chambers 5a arranged spaced apart in the direction of extent as well as with a projecting protrusion 5c.

FIG. 6 shows a detailed view of the upper half 4a of the handle 4. The lateral guide 4f shown at the bottom has grooves 4h which are spaced apart at regular intervals and into which the protrusion 5c of the reservoir 5 engages so that the reservoir 5 has latch positions defined with respect to the handle 4 in the direction of displacement B. These latch positions are selected such that a chamber 5a always comes to lie in the dispensing position A in which the fillers 2 present in the chamber 5a can be delivered to the cannula 3.

FIG. 9a shows a filler 2 in a plan view. In the embodiment shown, the filler is made as a hexagonal body with a cylindrical inner space. The filler could also have different outer contours. FIG. 9b shows four fillers 2 stacked over one another. In the representation in accordance with FIG. 5, as a rule at least some of the chambers 5a, and preferably all of the chambers 5a, are filled with the fillers 2 shown in FIGS. 7a and 7b so that the fillers 2 are arranged in each case sequentially in the direction of extent of each chamber 5a or next to one another. The fillers 2 are preferably arranged identically at opposite sides.

The fillers 2 located in the dispensing position in the chamber 5a can be expelled downwardly in the direction of extent of the chamber 5b into the cannula 3 located below it, for example via a plunger 7 acting perpendicular to the plane of observation. All the fillers 2 present in this chamber are thereby supplied group-wise to the cannula 3 and are pushed into the vertebral body 9.

The reservoir 5 shown in the FIGS. 7 and 8 preferably has between 4 and 20 chambers 5a spaced apart in the longitudinal direction. Depending on the height of the fillers 2 used, between 1 and 20 individual fillers 2, as shown in FIG. 9a, can be arranged in each chamber 5a. In a preferred embodiment, the inner cross-section of the chamber 5a is made in accordance with the outer contour of the fillers 2. In the embodiment shown, the outer contour of the filler 2 is hexagonal and the inner contour of the chamber 5a is likewise correspondingly hexagonal. All the chambers 5a are preferably filled with fillers 2 of the same size. It is, however, also possible to fill the chambers 5a with fillers 2 of different size, that is with a different diameter, a different outer contour and/or also a different height. It can prove to be advantageous for this purpose also to match the inner cross-section of the chamber 5a in accordance with the respective outer contour of the filler 2. The fillers 2 can be made in a plurality of possible outer contours, for example, also circular, elliptical, square, rectangular, etc.

FIG. 10 shows a front view and FIG. 11 shows a side view of a trocar 8 which includes a shaft 8a having a sharp, triangular tip and an actuation handle 8b. The trocar 8 moreover includes a fastening means 8c with the help of which the trocar 8 can be fastened via the cut-out 4e to the handle 4.

FIG. 12 shows a plunger 7 comprising a handle 7b as well as a shaft 7a having a plunger end 7c.

The total instrument 1 is preferably supplied in the arrangement shown in FIG. 13 in that the trocar 8 is fastened to the handle 4 with the help of the fastening means 8c and in that the shaft 8a extends through the access opening 4c, through the cylindrical chamber 5a and through the cannula 3 so that the tip of the shaft 8a projects out of the cannula tip 3a at the front. This arrangement has the additional advantage that the reservoir 5 cannot move in the direction of movement B due to the shaft 8a extending through the chamber 5b and is thus blocked and held in a defined position. Only after the complete removal of the trocar 8 from the instrument 1 can the reservoir 5 be displaced with respect to the handle 4.

As shown in FIG. 13, the instrument 1 can now, for example, be introduced into the human body such that the tip of the trocar 8 is introduced first. In the example shown, the trocar 8 is introduced into a vertebral body 9 only shown schematically. The cannula tip 3a is then placed onto the vertebral body 9 and the cannula 3 is screwed into the vertebral body 9 by turning in the direction of rotation C at the handle 4. The handle 4 is preferably made such that the handle 4 can also be actuated with the right hand or with both hands during this screw movement. As soon as the cannula 3 is arranged sufficiently deeply in the vertebral body 9, the trocar 8 is released from the handle 4 and is completely removed from the instrument 1. The reservoir 5 is thus unblocked and thereby displaceable in the handle 4. The handle 4 can now advantageously be held by the left hand. The first chamber 5a arranged subsequent to the chamber 5b is pushed into the dispensing position A by an actuation of the push button 6a. Thereupon, the plunger 7 is introduced by the right hand via the access opening 4c into the handle 4 and the fillers 2 present in the chamber 5a are pushed into the cannula 3 by the plunger 7. FIG. 3 schematically shows the chamber 5a present in the dispensing position A with fillers 2 which are pushed in the direction of the cannula 3 by the plunger 7.

FIG. 14 schematically shows a section through a vertebral body 9 in whose inner space 9a the fillers 2 are pushed in that the plunger 7 exerts a force F onto the fillers 2 present in the cannula 3 via the plunger end 7c. The number of the fillers 2 delivered simultaneously sequentially can be determined in that either in each case all the fillers 2 present in a chamber 5a are pushed into the vertebral body 9 or, after the first emptying of a chamber 5a, the plunger 7 is retracted, the push button 6a is actuated and the content of the subsequent chamber 5a is likewise pushed into the cannula 3 by the plunger 7. This procedure can, if desired, be repeated a plurality of times, in order, for example, to displace 2, 3, 5 or 10 contents of the chambers 5a into the cannula 3 first before all the fillers 2 sequentially arranged in the cannula 3 in this manner are pressed into the vertebral body 9 with the help of the plunger 7.

It can be found to be advantageous to provide a plurality of plungers 7 with, as shown in FIGS. 15a to 15e, differently made tips 7c. After the fillers 2 have been introduced in the vertebral body 9, their position can still partly be influenced in that a plunger 7 with a correspondingly shaped tip 7c is used in order, for example, to laterally displace the fillers 2 with the tip 7c shown in FIG. 15b. The position of the fillers 2 in the vertebral body 9 is preferably made visible with the help of an X-ray machine.

FIG. 16 schematically shows a further embodiment of a plunger 7 with whose help fillers 2 are supplied via an instrument 1 with a cannula 3 to a vertebral body 9. The plunger 7 shown includes a handle 7b, an active drive 7d, a sensor 7e, a regulation device 7f and a shaft 7a. The drive is, for example, made as an electrical motor drive or as a compressed air drive and preferably also enables a vibration movement in order, for example, to embed fillers 2 present in the vertebral body 9 in a compacted form. The regulation device 7f allows force or distance to be measured, for example with the sensor 7e, and the maximum thrust force or the maximum stroke of the vibration movement to be limited.

The cannula 3 can also have a curved extent, in particular if no screw movement is required for the fastening to the bone. The cannula 3 is preferably made of metal, but can also be made of a plastic. The plunger 7 can be made of metal or also of a plastic, in particular also of a resilient plastic.

The reservoir 5 can be made in different manners, for example also as a drum with chambers 5a arranged spaced apart in circular form in the peripheral direction respect to an axis of rotation, with the reservoir being mounted rotatably in the handle 4.

The fillers 2 can have a plurality of possible shapes such that the fillers 2 can be stored in the reservoir 5. The fillers 2 could, for example, also be spherical.

FIG. 17 shows an instrument 1 having a cannula 3, a handle 4 and a drum-shaped reservoir 5 which is rotatably mounted on the handle 4 in the indicated direction of rotation and has a chamber located in the dispensing position 5b and a plurality of further chambers 5a arranged spaced apart in the peripheral direction for the reception of fillers 2. The reservoir 5 has grooves at the cylindrical outer peripheral surface via which grooves the position of the reservoir 5 can be rotated manually. The instrument 1 preferably has a latch device so that the chambers 5a are respectively located in the dispensing position 5b after a rotation.

FIG. 18 shows a plan view of the reservoir 5 shown in FIG. 17. The reservoir is rotatably mounted around an axis of rotation 5e.

FIG. 19 shows a further embodiment of an instrument 1. Unlike the embodiment shown in FIG. 18, this instrument 1 has a push button 6a, with the push button 6a and the reservoir 5 being made such that, when the push button 6a is actuated, the reservoir 5 is rotated in each case around a position in the direction drawn so that sequential chambers 5a are conveyed into the dispensing position 5b so that the fillers 2 present in the chamber 5a can be delivered to the cannula 3 using the plunger 7.

The instrument 1 is preferably made as a disposable instrument. It can, however, also prove to be advantageous to use the instrument 1 a plurality of times and only to replace the reservoir 5 with a new reservoir 5 filled with fillers 2 after the use of the instrument 1.

It can prove to be advantageous to heat the fillers 2 before the introduction into the vertebral body, for example to a temperature between 50 and 70 degrees C., or up to 100 degrees C. This can be achieved, for example, in that the reservoir 5 filled with fillers 2 is heated before the reservoir 5 is introduced into the instrument 1. It can also prove to be advantageous to provide the instrument 1 with a heating, for example with an electrical heating, to heat the fillers 2 present in the instrument 1. It can moreover prove to be advantageous to heat the cannula 3.

1. An instrument (1) for delivery of solid fillers (2) into an inner space of a bone, comprising: a rigid cannula (3) coupled to a handle (4), and a reservoir (5) movably coupled to the handle (4), wherein the reservoir (5) has a plurality of chambers (5a) that are configured to receive solid fillers (2), wherein the chambers (5a) are arranged in the reservoir (5) such that the chambers (5a) can be sequentially positioned in a dispensing position (A) with respect to the cannula (3) by moving of the reservoir (5) so that the fillers (2) can be delivered to the chamber (5b) of the cannula (3) respectively located in the dispensing position (A).

2. The instrument in accordance with claim 1, characterized in that the handle (4) has an access opening (4c) that extends in a direction of the cannula (3); and in that the dispensing position (A) is located between the cannula (3) and the access opening (4c).

3. The instrument in accordance with claim 1, characterized in that the reservoir (5) has a bar shape having a plurality of chambers spaced (5a) along a longitudinal axis of the reservoir.

4. The instrument in accordance with claim 3, characterized in that the reservoir (5) is disposed within the handle (4) along a longitudinal axis of the handle.

5. The instrument in accordance with claim 1, characterized in that the reservoir (5) has a drum shape having chambers (5a) circularly arranged therein.

6. The instrument in accordance with claim 1, characterized in that a feed part (6) moves the reservoir (5) such that a chamber (5b) respectively containing a filler (2) is moved to the dispensing position (A) after the actuation of the feed part (6).

7. The instrument in accordance with claim 6, characterized in that the feed part (6) includes a push button (6a) which is disposed with respect to the handle (4) such that, when a hand encloses the handle (4), the push button (6a) can be actuated by the thumb of the hand.

8. The instrument in accordance with claim 2, further comprising a plunger (7) that is configured to allow introduction of a portion of the plunger into the cannula (3) via the access opening (4c) such as to allow exertion of a plunging force on fillers located in the dispensing position (A) or in the cannula (3).

9. The instrument in accordance with claim 1, further comprising a trocar (8) having a shaft (8a), with the shaft (8a) being configured such as to allow delivery of the shaft into the cannula (3) via the access opening (4c).

10. The instrument in accordance with claim 9, characterized in that the trocar (8) is releasably connected to the handle (4); and in that the shaft (8a) of the trocar (8) extends through the access opening (4c), through an empty chamber (5a) of the reservoir (5) as well as through the cannula (3).

11. The instrument in accordance with claim 9, characterized in that the trocar (8) is releasably connected to the handle (4) via a fastening means (8c).

12. The instrument in accordance with claim 1, characterized in that the chambers (5a) contain fillers (2).

13. The instrument in accordance with claim 12, characterized in that the chambers (5a) have a longitudinal direction; in that the chambers (5a) are made matched to one another such that a plurality of fillers (2) are respectively arranged in a chamber (5a); and in that the fillers (2) are disposed sequentially in the longitudinal direction of the chambers (5a).

14. The instrument in accordance with claim 13, characterized in that the fillers (2) have an angular outer contour; and in that the chambers (5a) have an inner cross-section adapted to the outer contour of the filler (2).

15. The instrument in accordance with claim 13, characterized in that each chamber (5a) contains between 1 and 20 fillers (2); and in that each reservoir contains between 4 and 20 chambers (5a).

16. A reservoir (5) for an instrument (1) in accordance with claim 1 wherein a plurality of chambers (5a) are filled with fillers (2), wherein the chambers (5a) have a longitudinal direction, and wherein the chambers (5a) and the fillers (2) are mutually matched to one another such that a plurality of fillers (2) are in each case arranged in a chamber (5a); and in that the fillers (2) are arranged disposed sequentially in the direction of extent of the chambers (5a).

17. The reservoir in accordance with claim 16, characterized in that all fillers (2) are aligned identically in the chambers (5a).

18. A kit comprising an instrument in accordance with claim 1, a reservoir (5) having fillers (2) in accordance with claim 16, and at least one plunger (7) suitable for introduction of the fillers (2).

19. The kit in accordance with claim 18 comprising a plurality of plungers (7) having differently configured tips (7c).

20. The kit in accordance with claim 18, further comprising a vibration device that is configured to functionally cooperate with the plunger (7).
